(19) United States
(12) Patent Application Publication
(10) Pub. No.: US 2006/0070191 A1
(43) Pub. Date: Apr. 6, 2006

(54) Composition for dyeing keratinous fibres with a cationic direct dye and a nonionic surfactant

(75) Inventors: Gerard Lang, Saint Prix (FR); Jean Cotteret, Verneuil Sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/121,156

(22) Filed: May 4, 2005

Related U.S. Application Data
(63) Continuation of application No. 09/530,015, filed on May 25, 2000, now abandoned.

(30) Foreign Application Priority Data
Aug. 24, 1998 (FR) 98/10659

(51) Int. Cl. A61K 8/49; A61K 8/41

(52) U.S. Cl. 8/406

(57) ABSTRACT

The invention relates to a composition for dyeing keratinous fibres, in particular human keratinous fibres such as hair, comprising, in an appropriate dyeing medium, at least one cationic direct dye of a given formula, and which is characterized in that it contains, in addition, at least one nonionic surfactant chosen from the group consisting of alkyl polyglucosides, sugar or alkyl sugar fatty acid esters, fatty sugar amides and mixtures thereof. The invention also relates to the dyeing methods and devices using it.

The invention relates to a composition for dyeing keratinous fibres, in particular human keratinous fibres such as hair, comprising, in an appropriate dyeing medium, at least one cationic direct dye of a given formula, and at least one particular nonionic surfactant.

The subject of the invention is also the dyeing methods and devices using the said composition.

In the hair domain, it is possible to distinguish two types of dyeing.

The first is the semipermanent or temporary dyeing, or direct dyeing, which involves dyes capable of bringing the natural colour of the hair a more or less marked colour modification which is resistant, where appropriate, to several shampooings. These dyes are called direct dyes; they can be used with or without oxidizing agent. In the presence of oxidizing agent, the aim is to obtain a lightening dyeing. Lightening dyeing is performed by applying to the hair the fresh mixture of a direct dye and of an oxidizing agent and makes it possible in particular to obtain, by lightening of the melanin of the hair, an advantageous effect such as a uniform colour in the case of grey hair or to make the colour stand out in the case of naturally pigmented hair.

The second is permanent dyeing or oxidation dyeing. The latter is performed with so-called “oxidation” dyes comprising oxidation dye precursors and couplers. The oxidation dye precursors, commonly called “oxidation bases” are compounds which are initially colourless or faintly coloured which develop their dyeing power inside the hair in the presence of oxidizing agents added at the time of use, leading to the formation of coloured and dyeing compounds. The formation of these coloured and dyeing compounds results either from an oxidative condensation of the “oxidation bases” with themselves, or an oxidative condensation of the “oxidation bases” with colour modifying compounds commonly called “couplers” and generally present in the dyeing compositions used in oxidation dyeing.

To vary the shades obtained with the said oxidation dyes, or to increase their shimmer, direct dyes are sometimes added to them.

Among the cationic direct dyes available in the field of dyeing of keratinous fibres, especially human keratinous fibres, compounds are already known whose structure is developed in the text which follows; nevertheless, these dyes lead to colours which exhibit characteristics which are still inadequate from the point of view of the intensity and homogeneity of the colour distributed along the fibre; it is said, in this case, that the colour is too selective, and from the point of view of fastness, in terms of resistance to various attacks to which the hair may be subjected (light, adverse weather conditions, shampooings).

However, after major research studies carried out on this question, the applicant has just now discovered that it is possible to obtain novel compositions for dyeing keratinous fibres which are capable of giving intense and only slightly selective colours which are quite resistant nevertheless to the various attacks to which the hair may be subjected, by combining at least one particular anionic surfactant with at least one cationic direct dye known in the prior art and which have the respective formulae defined hereinafter.

This discovery forms the basis of the present invention.

The first subject of the present invention is therefore a composition for dyeing keratinous fibres and in particular human keratinous fibres such as hair, containing in an appropriate dyeing medium, (i) at least one cationic direct dye whose structure corresponds to the formulae (I) to (IV) defined hereinafter, characterized in that it contains in addition (ii) at least one particular nonionic surfactant.

(i) The cationic direct dye which can be used according to the present invention is a compound chosen from those of the following formulae (I), (II), (III), (III′), (IV):

a) the compounds of the following formula (I): 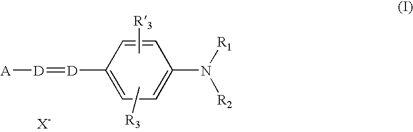
in which:

D represents a nitrogen atom-or the —CH group,

R₁ and R₂, which are identical or different, represent a hydrogen atom; a C₁-C₄ alkyl radical which may be substituted with a —CN, —OH or —NH₂ radical or form with a carbon atom of-the benzene ring an optionally oxygen-containing or nitrogen-containing heterocycle which may be substituted with one or more C₁-C₄ alkyl radicals; a 4′-aminophenyl radical,

R₃ and R′₃, which are identical or different, represent a hydrogen or halogen atom chosen from chlorine, bromine, iodine and fluorine, a cyano, C₁-C₄ alkyl, C₁-C₄ alkoxy or acetyloxy radical,

X⁻ represents an anion which is preferably chosen from chloride, methylsulphate and acetate, A represents a group chosen from the following structures A₁ to A₁₉: 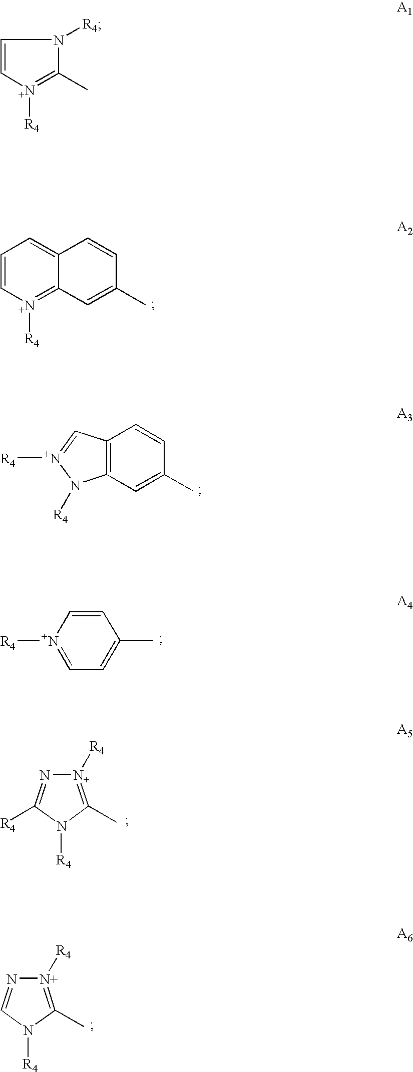 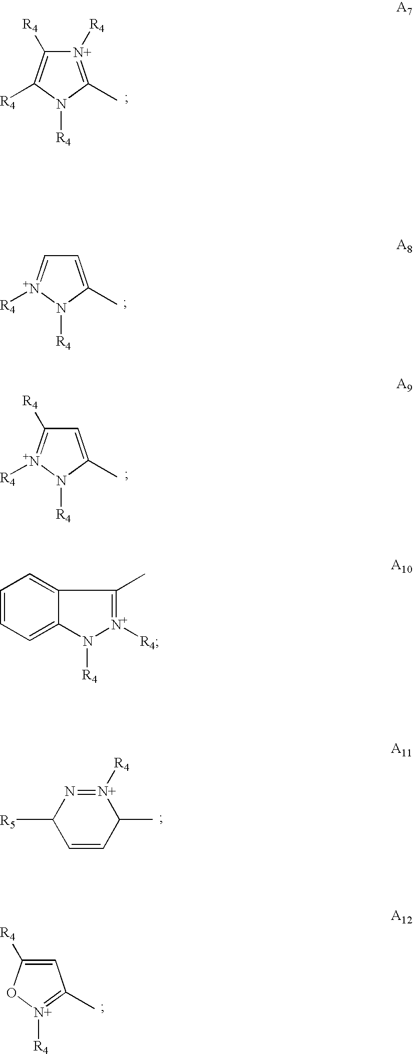 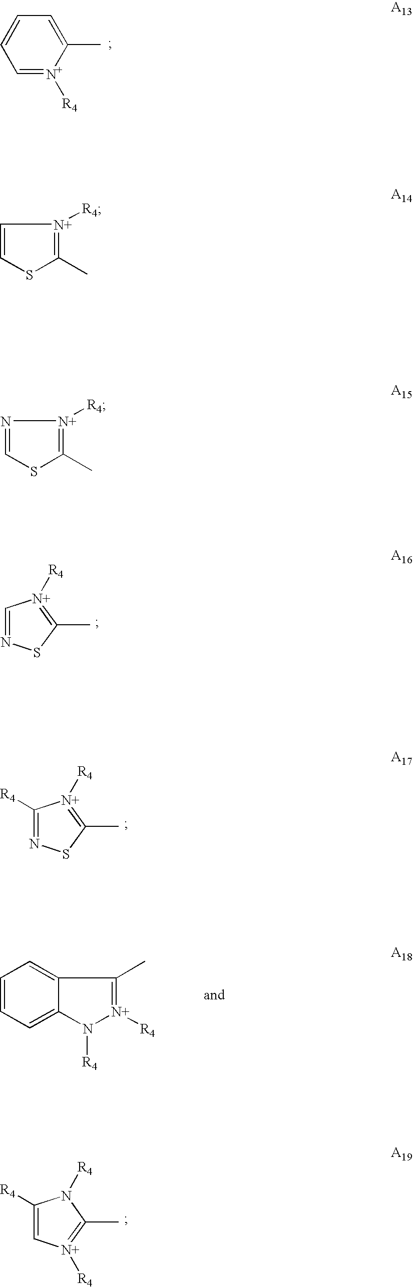
in which R₄ represents a C₁-C₄ alkyl radical which may be substituted with a hydroxyl radical and R₅ represents a C₁-C₄ alkoxy radical, with the proviso that when D represents —CH, A represents A₄ or A₁₃ and R₃ is different from an alkoxy radical, then R₁ and R₂ do not simultaneously denote a hydrogen atom;

b) the compounds of the following formula (II): 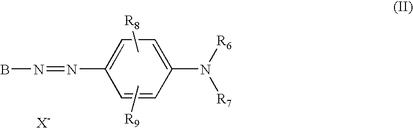
in which:

R₆ represents a hydrogen atom or a C₁-C₄ alkyl radical,

R₇ represents a hydrogen atom, an alkyl radical which may be substituted with a —CN radical or with an amino group, a 4′-aminophenyl radical or forms with R₆ an optionally oxygen-containing and/or nitrogen-containing heterocycle which may be substituted with a C₁-C₄ alkyl radical,

R₈ and R₉, which are identical or different, represent a hydrogen atom, a halogen atom such as bromine, chlorine, iodine or fluorine, a C₁-C₄ alkyl or C₁-C₄ alkoxy radical, a —CN radical,

X⁻ represents an anion which is preferably chosen from chloride, methylsulphate and acetate,

B represents a group chosen from the following structures B1 to B6: 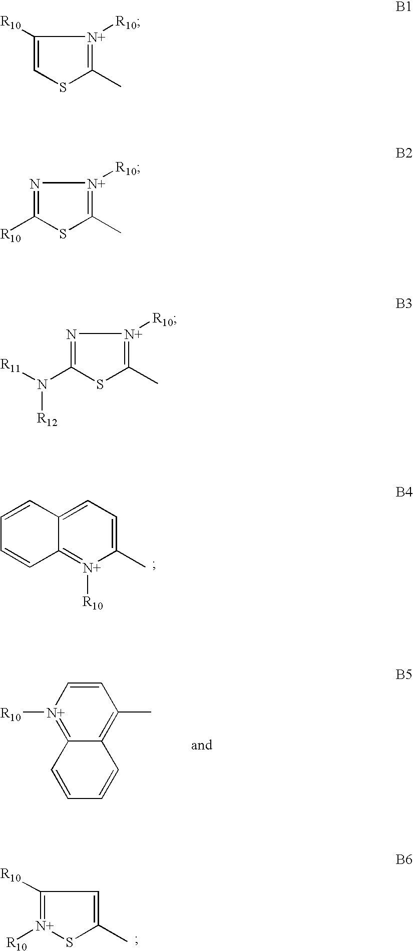
in which R₁₀ represents a C₁-C₄ alkyl radical, R₁₁ and R₁₂, which are identical or different, represent a hydrogen atom or a C₁-C₄ alkyl radical;

c) the compounds of the following formulae (III) and (III′): 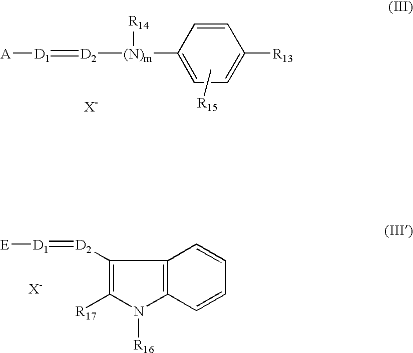
in which:

R₁₃ represents a hydrogen atom, a C₁-C₄ alkoxy radical, a halogen atom such as bromine, chlorine, iodine or fluorine or an amino radical,

R₁₄ represents a hydrogen atom, a C₁-C₄ alkyl radical or forms with a carbon atom of the benzene ring a heterocycle which is optionally oxygen-containing and/or substituted with one or more C₁-C₄ alkyl groups,

R₁₅ represents a hydrogen or halogen atom such as bromine, chlorine, iodine of fluorine,

R₁₆ and R₁₇, which are identical or different, represent a hydrogen atom or a C₁-C₄ alkyl radical,

D₁ and D₂, which are identical or different, represent a nitrogen atom or the —CH group,
m=0 or 1,
it being understood that when R₁₃ represents an unsubstituted amino group, then D₁ and D₂ simultaneously represent a —CH group and m=0,

X⁻ represents an anion which is preferably chosen from chloride, methylsulphate and acetate,

E represents a group chosen from the following structures E1 to E8: 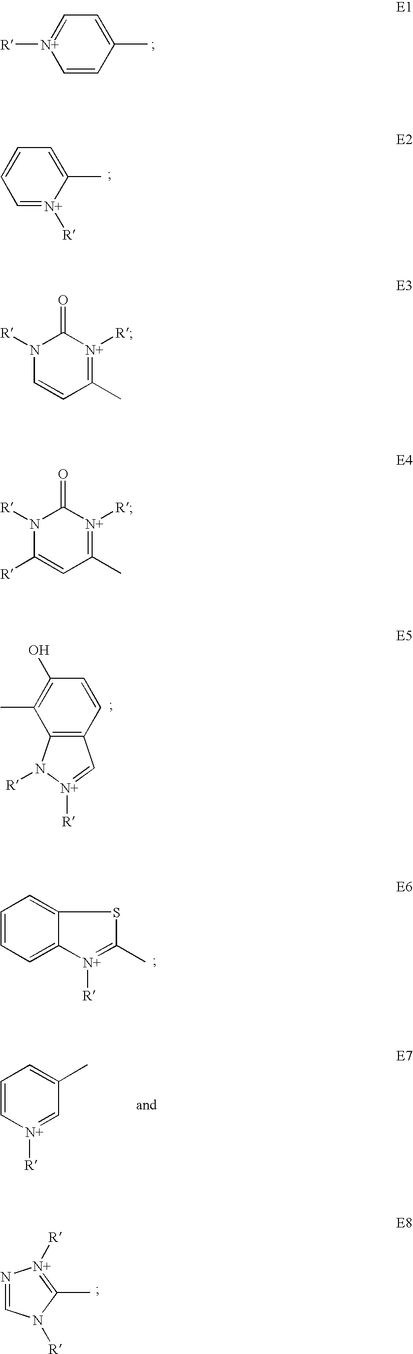
in which R′ represents a C₁-C₄ alkyl radical;

when m=0 and D₁ represents a nitrogen atom, then E may also denote a group having the following structure E9: 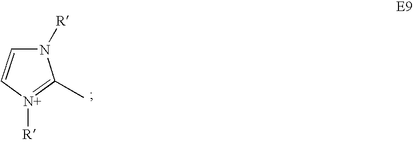
in which R′ represents a C₁-C₄ alkyl radical,

d) the compounds of the following formula (IV): 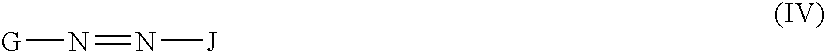
in which:

the symbol G represents a group chosen from the following structures G₁ to G₃: 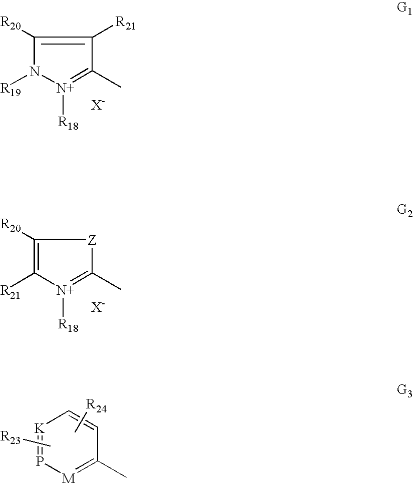
in which structures G₁ to G₃,

R₁₈ denotes a C₁-C₄ alkyl radical, a phenyl radical which may be substituted with a C₁-C₄ alkyl radical or a halogen atom chosen from chlorine, bromine, iodine and fluorine;

R₁₉ denotes a C₁-C₄ alkyl radical or a phenyl radical;

R₂₀ and R₂₁, which are identical or different, represent a C₁-C₄ alkyl radical, a phenyl radical, or form together in G₁ a benzene ring which is substituted with one or more C₁-C₄ alkyl, C₁-C₄ alkoxy or NO₂ radicals, or form together in G₂ a benzene ring which is optionally substituted with one or more C₁-C₄ alkyl, C₁-C₄ alkoxy or NO₂ radicals;

R₂₀ may denote, in addition, a hydrogen atom;

Z denotes an oxygen or sulphur atom or an —NR₁₉ group;

M represents a group —CH, —CR (R denoting C₁-C₄ alkyl), or —NR₂₂ (X⁻)ᵣ;

K represents a group —CH, —CR (R denoting C₁-C₄ alkyl), or —NR₂₂ (X⁻)ᵣ;

P represents a group —CH, —CR (R denoting C₁-C₄ alkyl), or —NR₂₂(X⁻)ᵣ; r denotes zero or 1;

R₂₂ represents an O⁻ atom, a C₁-C₄ alkoxy radical or a C₁-C₄ alkyl radical;

R₂₃ and R₂₄, which are identical or different, represent a hydrogen or halogen atom chosen from chlorine, bromine, iodine and fluorine, a C₁-C₄ alkyl radical, a C₁-C₄ alkoxy radical or an —NO₂ radical;

X⁻ represents an anion which is preferably chosen from chloride, iodide, methylsulphate, ethylsulphate, acetate and perchlorate;
with the proviso that

if R₂₂ denotes O⁻, then r denotes zero;

if K or P or M denote —N—(C₁-C₄ alkyl)X⁻, then R₂₃ or R₂₄ is different from a hydrogen atom;

if K denotes —NR₂₂(X⁻)_{r′}, then M=P═—CH, —CR;

if M denotes —NR₂₂(X⁻)ᵣ, then K═P═—CH, —CR;

if P denotes —NR₂₂(X⁻)ᵣ, then K=M and denote —CH or —CR;

if Z denotes a sulphur atom with R₂₁ denoting C₁-C₄ alkyl, then R₂₀ is different from a hydrogen atom;

if Z denotes —NR₂₂ with R₁₉ denoting C₁-C₄ alkyl, then at least one of the R₁₈, R₂₀ or R₂₁ radicals of the group having the structure G₂ is different from a C₁-C₄ alkyl radical;

the symbol J represents:

(a) a group having the following structure J₁: 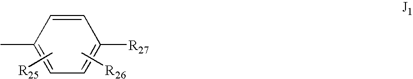
in which structure J₁,

R₂₅ represents a hydrogen atom, a halogen atom chosen from chlorine, bromine, iodine and fluorine, a C₁-C₄ alkyl radical, a C₁-C₄ alkoxy radical, a radical —OH, —NO₂, —NHR₂₈, —NR₂₉R₃₀, —NHCO(C₁-C₄alkyl), or forms with R₂₆ a 5- or 6-membered ring containing or otherwise one or more heteroatoms chosen from nitrogen, oxygen or sulphur;

R₂₆ represents a hydrogen atom, a halogen atom chosen from chlorine, bromine, iodine and fluorine, a C₁-C₄ alkyl or C₁-C₄ alkoxy radical, or forms with R₂₇ or R₂₈ a 5- or 6-membered ring containing or otherwise one or more heteroatoms chosen from nitrogen, oxygen or sulphur;

R₂₇ represents a hydrogen atom, an —OH radical, an —NHR₂₈ radical, an —NR₂₉R₃₀ radical;

R₂₈ represents a hydrogen atom, a C₁-C₄ alkyl radical, a C₁-C₄ monohydroxyalkyl radical, a C₂-C₄ polyhydroxyalkyl radical, a phenyl radical;

R₂₉ and R₃₀, which are identical or different, represent a C₁-C₄ alkyl radical, a C₁-C₄ monohydroxyalkyl radical, a C₂-C₄ polyhydroxyalkyl radical;

(b) a 5- or 6- membered nitrogen-containing heterocycle group which is capable of containing other heteroatoms and/or carbonyl-containing groups and which may be substituted with one or more C₁-C₄ alkyl, amino or phenyl radicals,

and in particular a group having the following structure J₂: 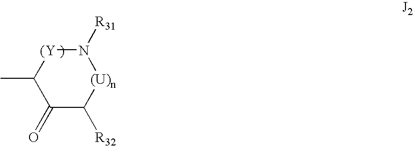
in which structure J₂,

R₃₁ and R₃₂, which are identical or different, represent a hydrogen atom, a C₁-C₄ alkyl radical, a phenyl radical;

Y denotes the —CO— radical or the radical 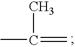
n=0 or 1, with, when n denotes 1, U denotes the —CO— radical.

In the structures (I) to (IV) defined above, the C₁-C₄ alkyl or alkoxy group preferably denotes methyl, ethyl, butyl, methoxy or ethoxy.

The cationic direct dyes of formulae (I), (II), (III) and (III′) which can be used in the dyeing compositions in accordance with the invention are known compounds which are described, for example, in patent applications WO 95/01772, WO 95/15144 and EP-A-0,714,954. Those of formula (IV) which can be used in the dyeing compositions in accordance with the invention are known compounds which are described, for example, in patent applications FR-2,189,006, FR-2,285,851 and FR-2,140,205 and its certificates of addition.

Among the cationic direct dyes of formula (I) which can be used in the dyeing compositions in accordance with the invention, there may be mentioned more particularly the compounds corresponding to the following structures (I1) to (I54):

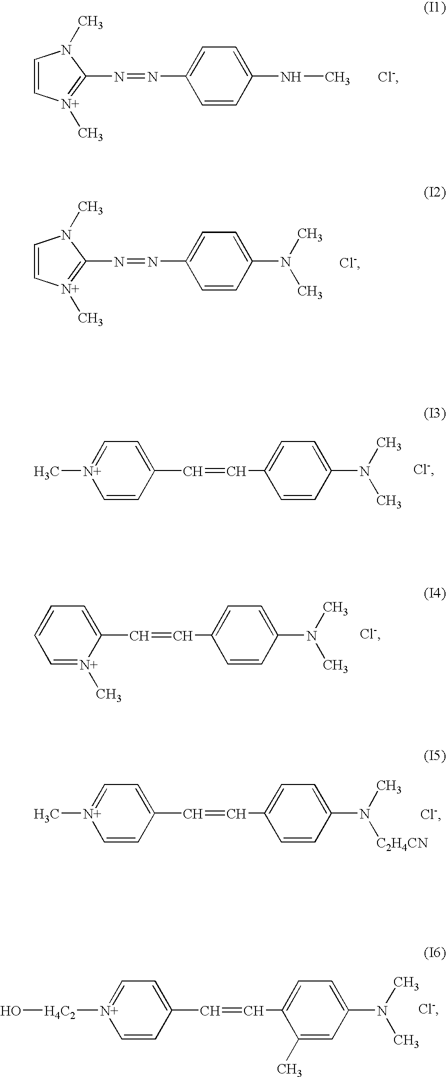

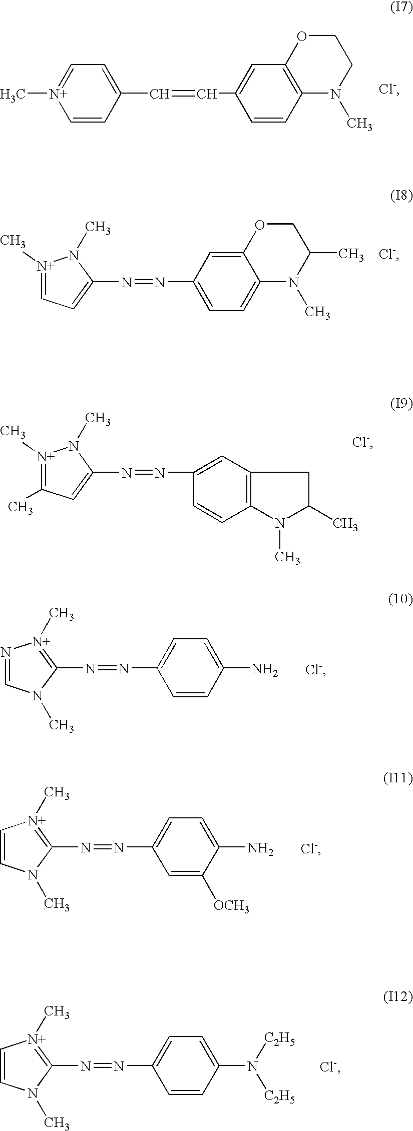

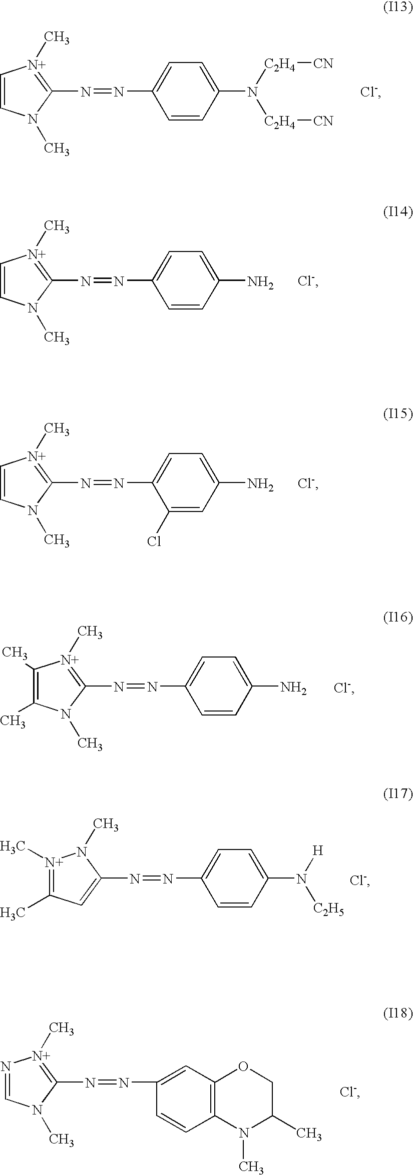

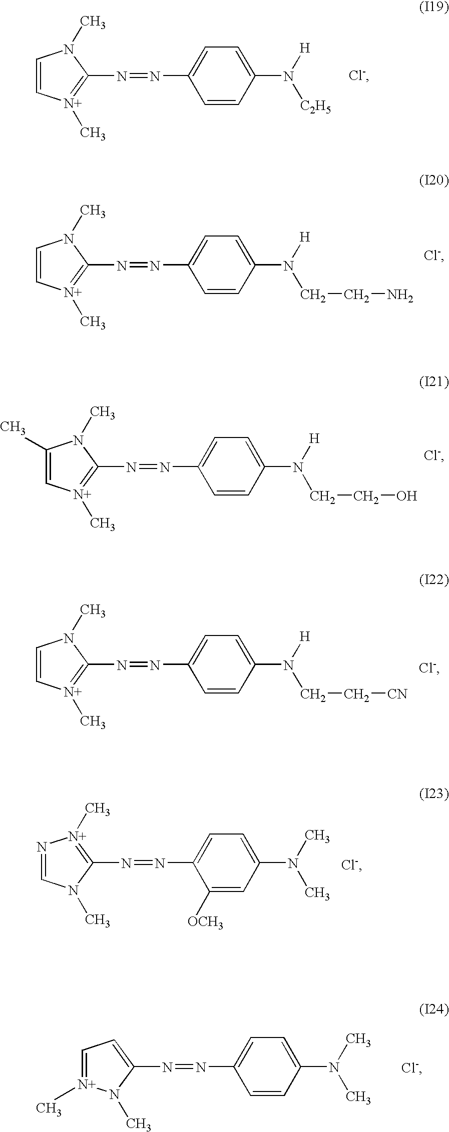

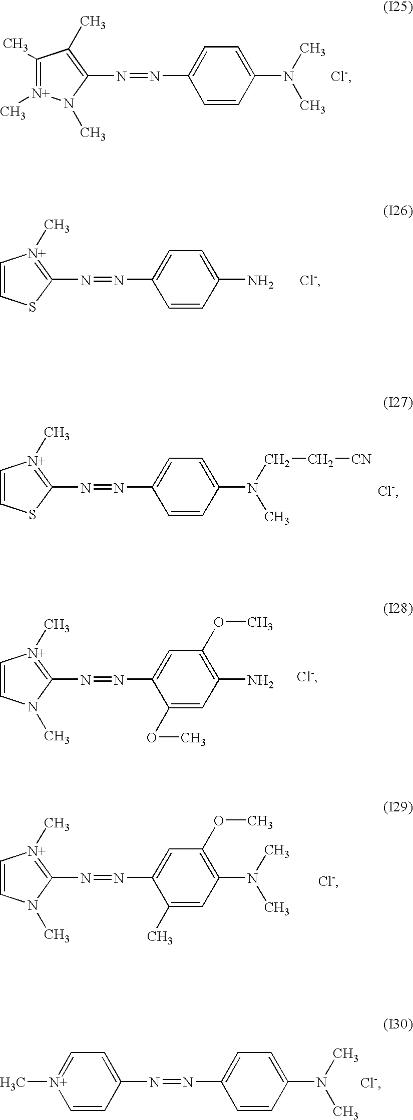

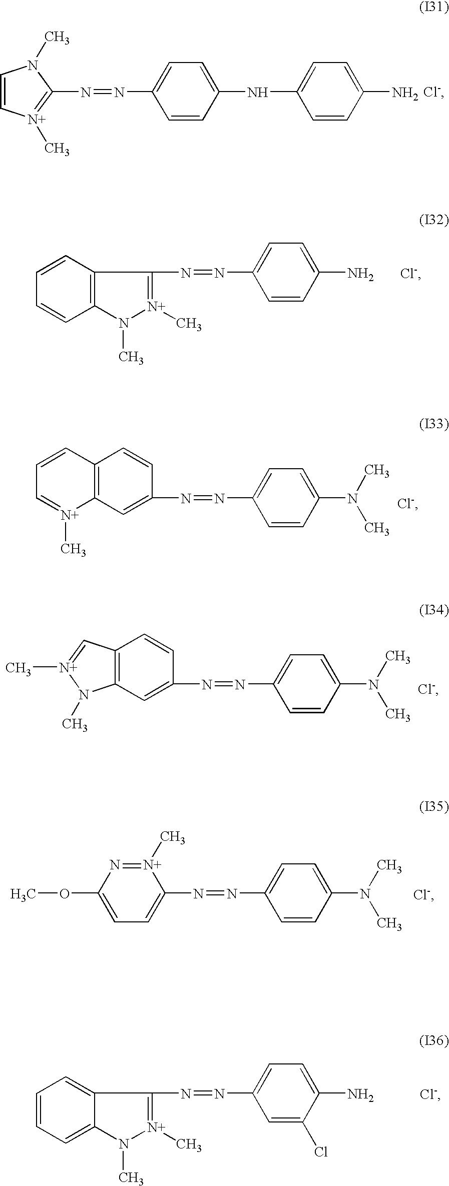

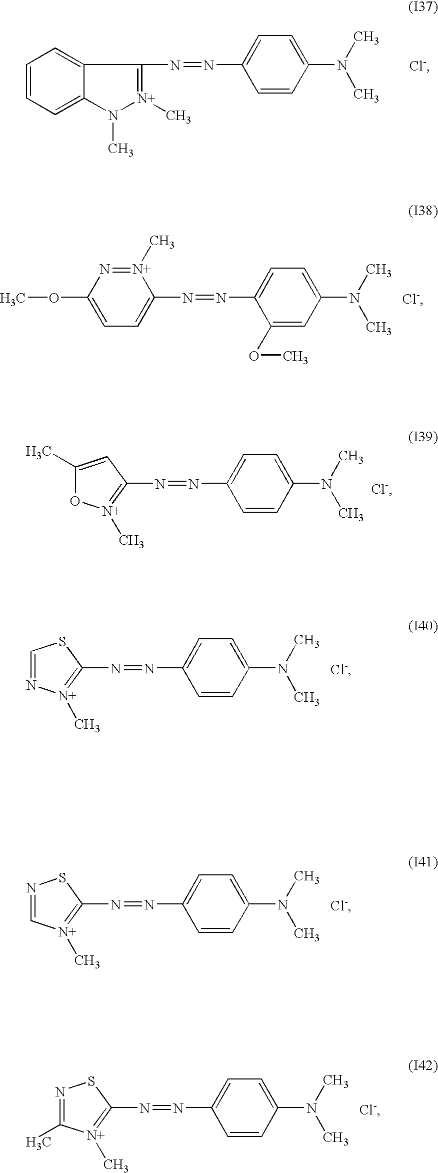

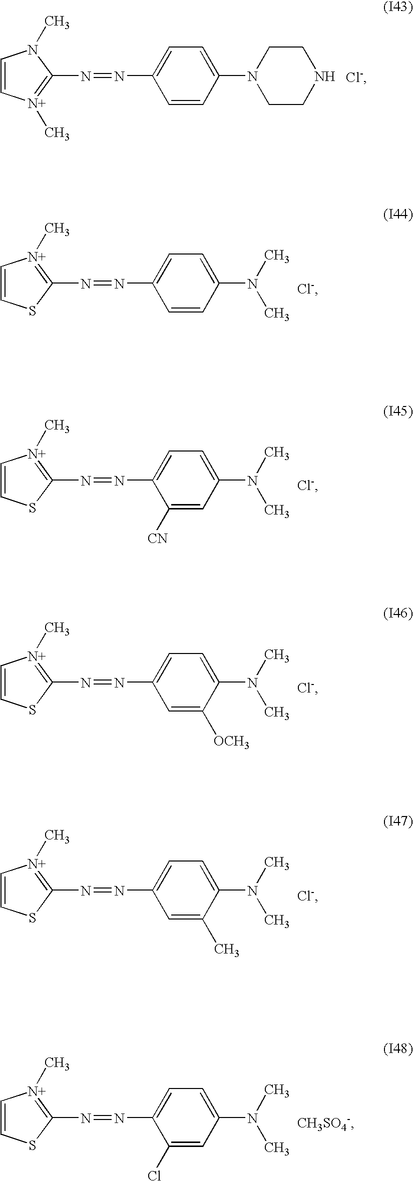

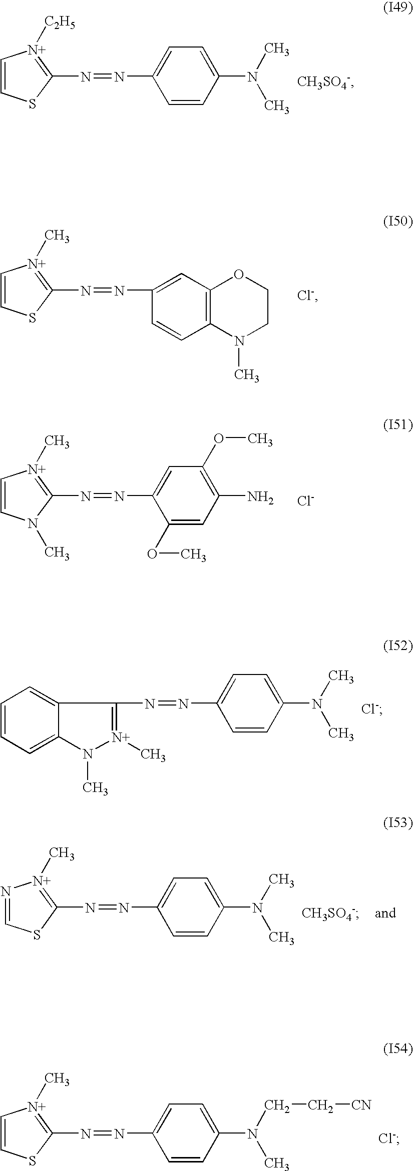

Among the compounds having the structures (I1) to (I54) which are described above, the compounds corresponding to the structures (I1), (I2), (I14) and (I31) are most particularly preferred.

Among the cationic direct dyes of formula (II) which can be used in the dyeing compositions in accordance with the invention, there may be mentioned more particularly the compounds corresponding to the following structures (II1) to (II9):

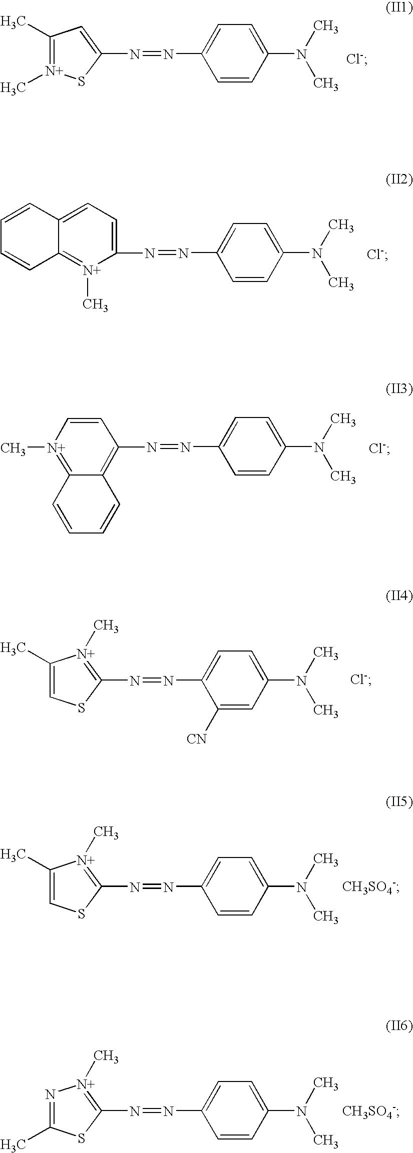

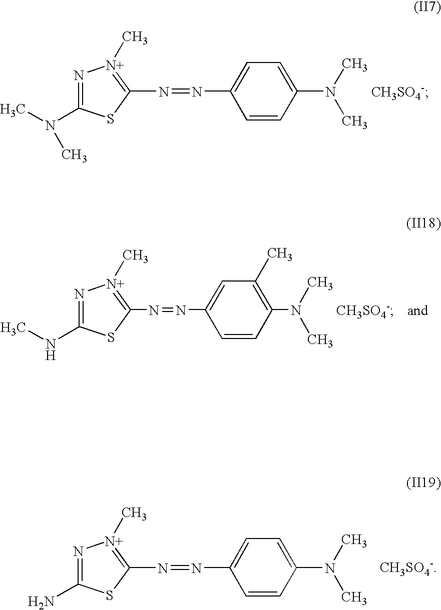

Among the cationic direct dyes of formula (III) which can be used in the dyeing compositions in accordance with the invention, there may be mentioned more particularly the compounds corresponding to the following structures (III1) to (III18):

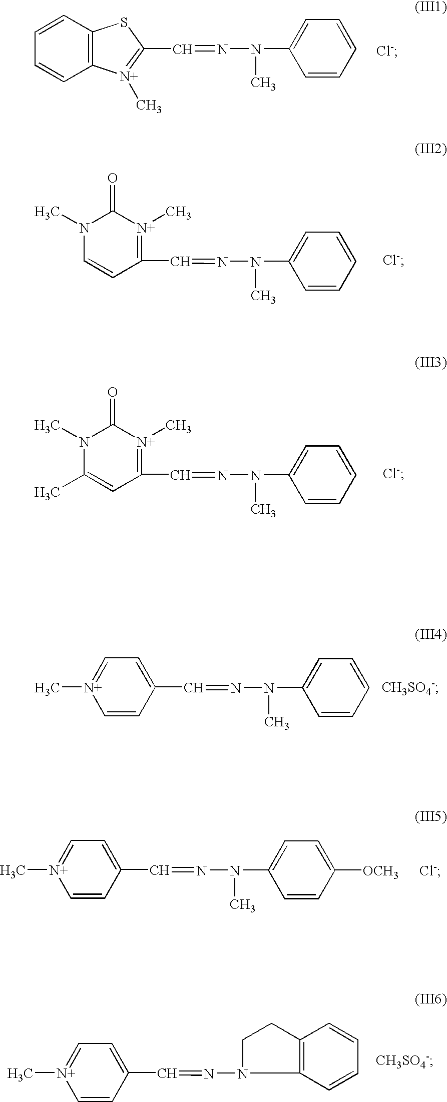

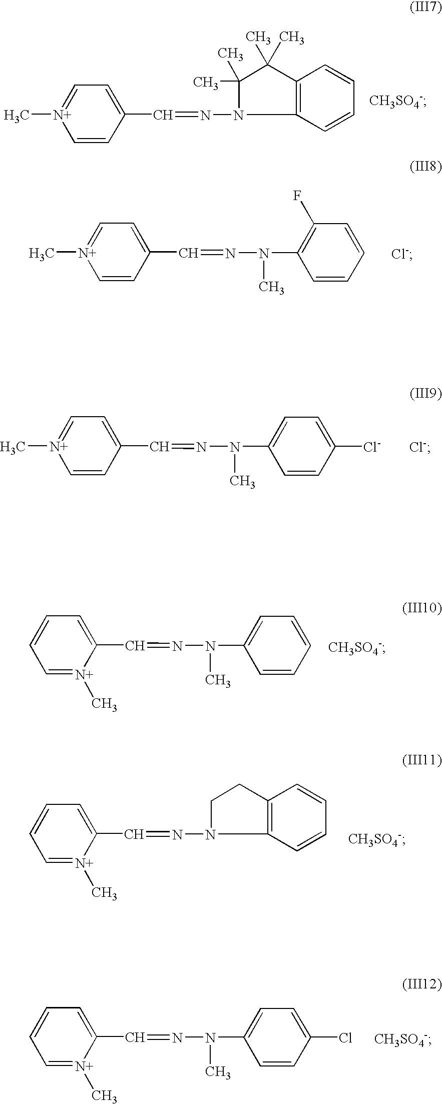

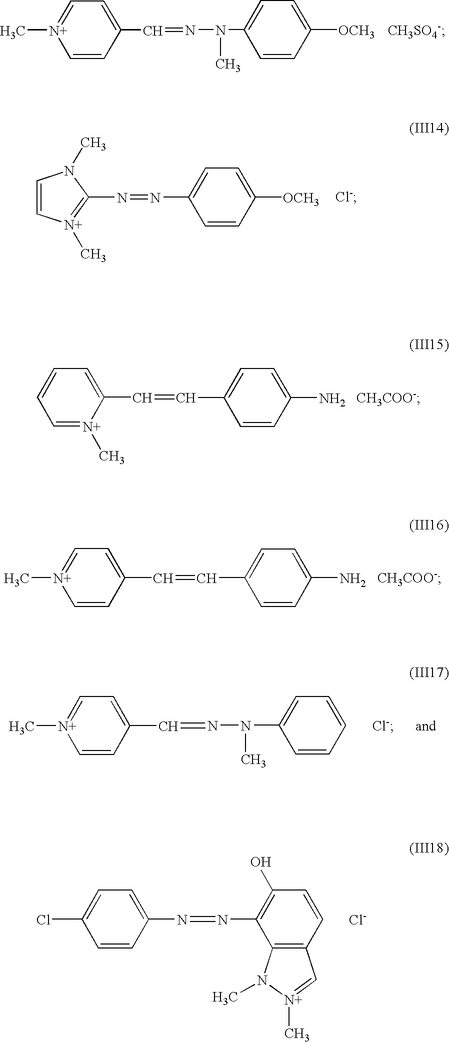

Among the particular compounds having the structures (III1) to (III18) which are described above, the compounds corresponding to the structures (III4), (III5) and (III13) are most particularly preferred.

Among the cationic direct dyes of formula (III′) which can be used in the dyeing compositions in accordance with the invention, there may be mentioned more particularly the compounds corresponding to the following structures (III′1) to (III′3):

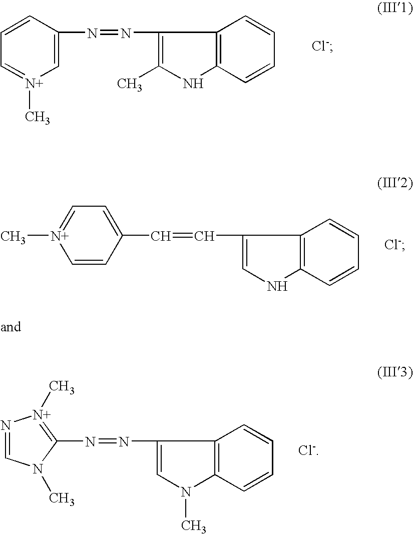

Among the cationic direct dyes of formula (IV) which can be used in the dyeing compositions in accordance with the invention, there may be mentioned more particularly the compounds having the following structures (IV)₁ to (IV)₇₇:

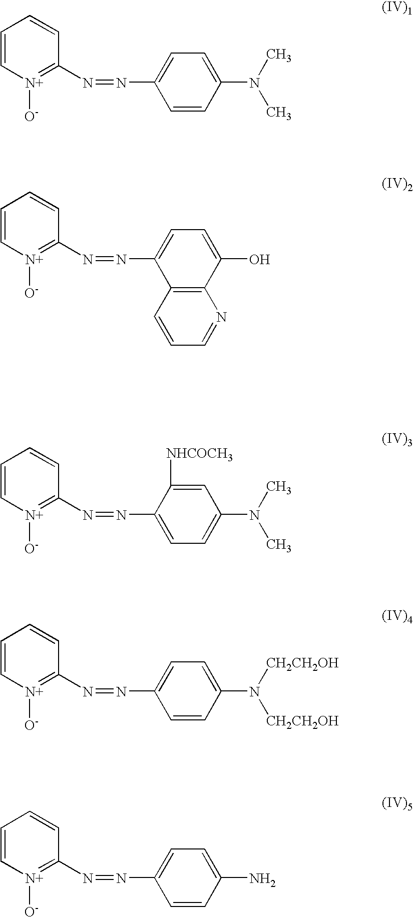

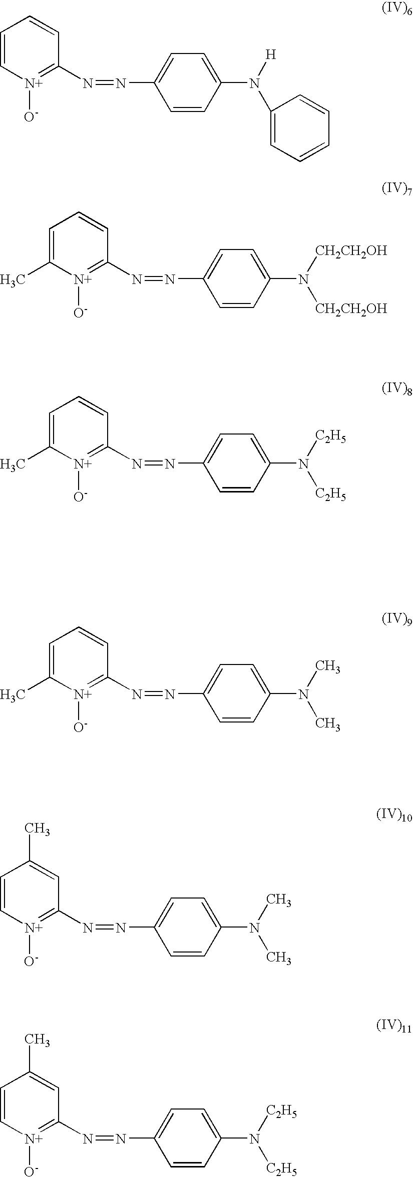

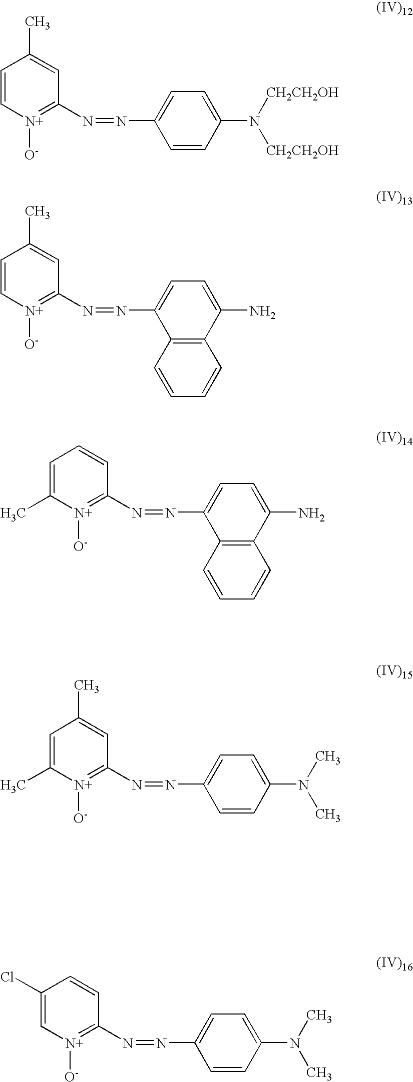

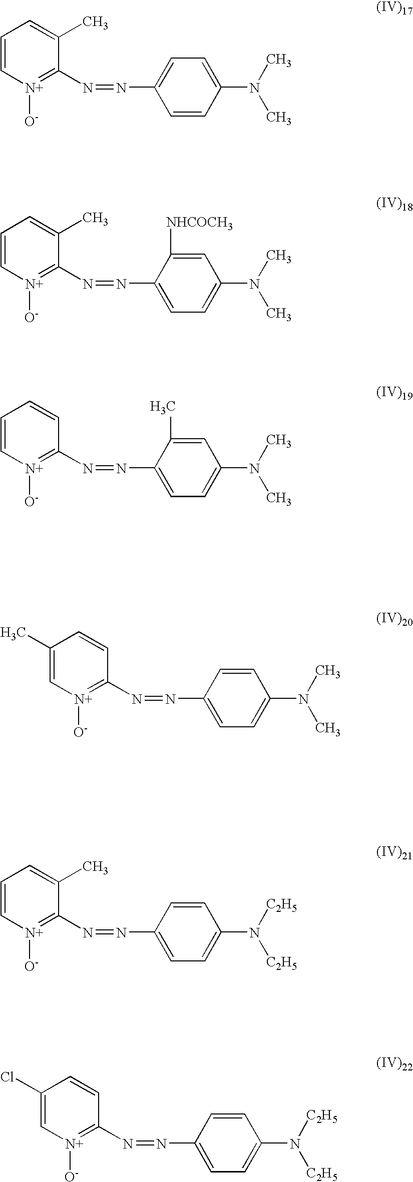

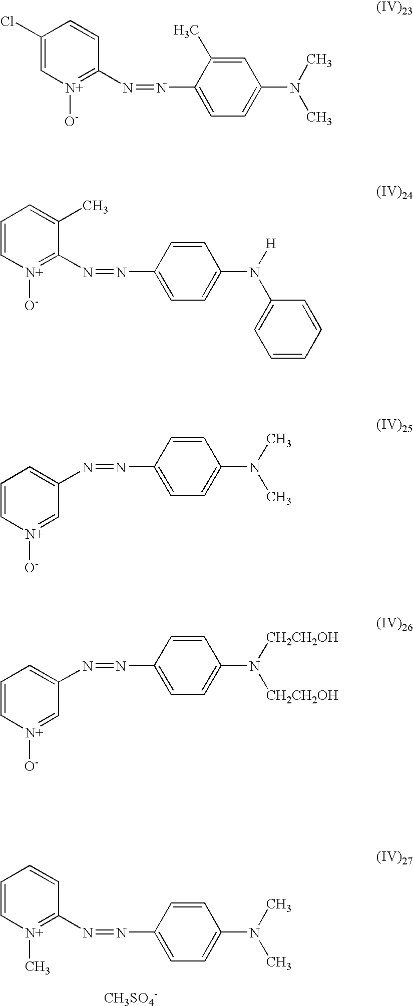

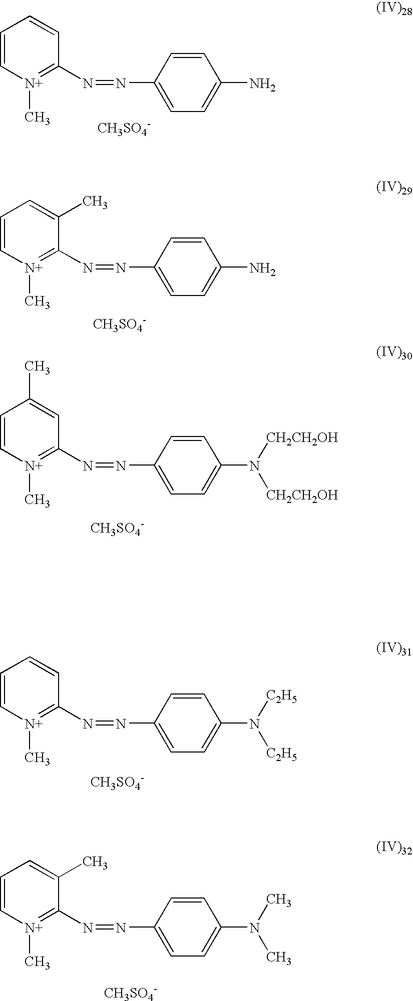

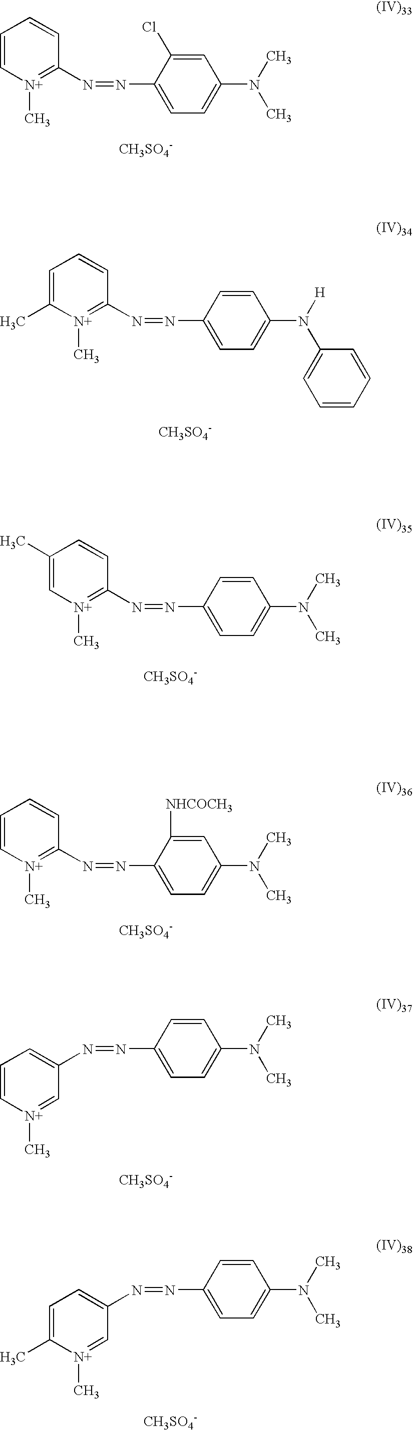

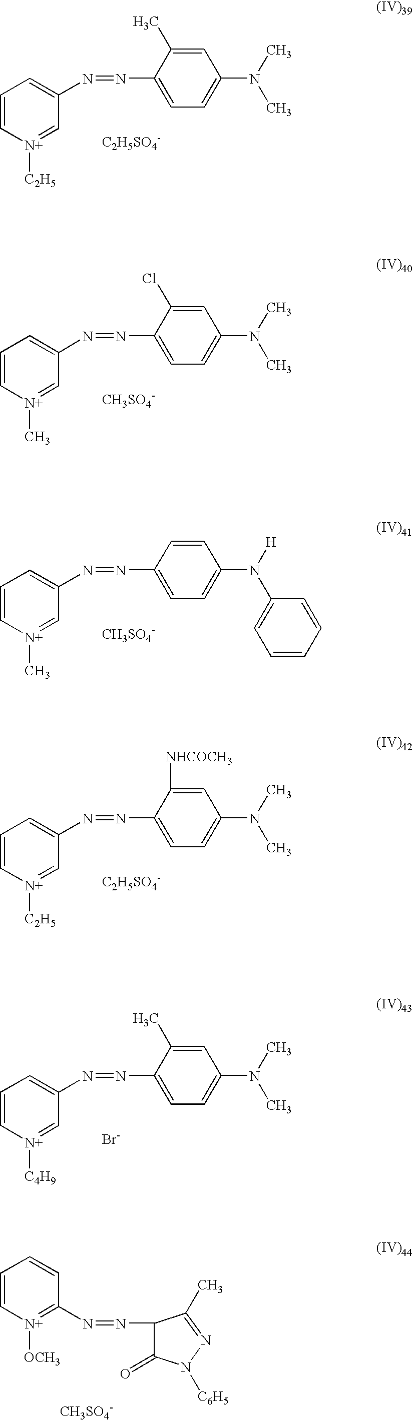

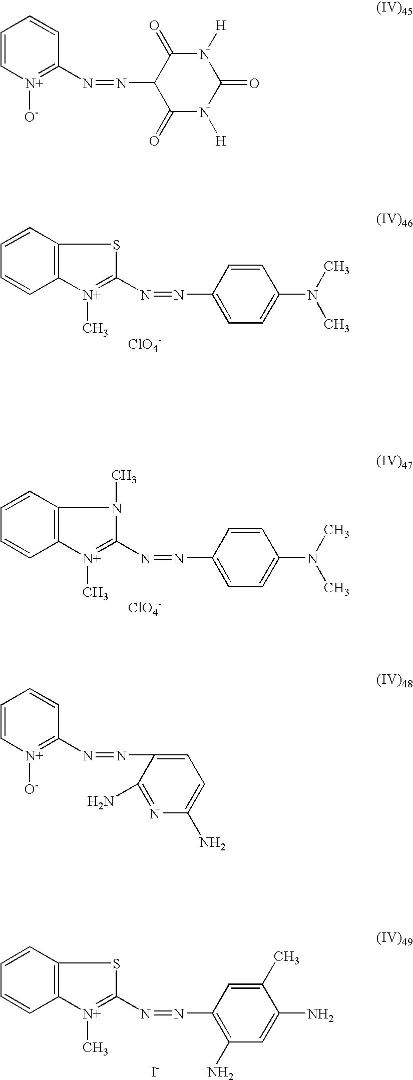

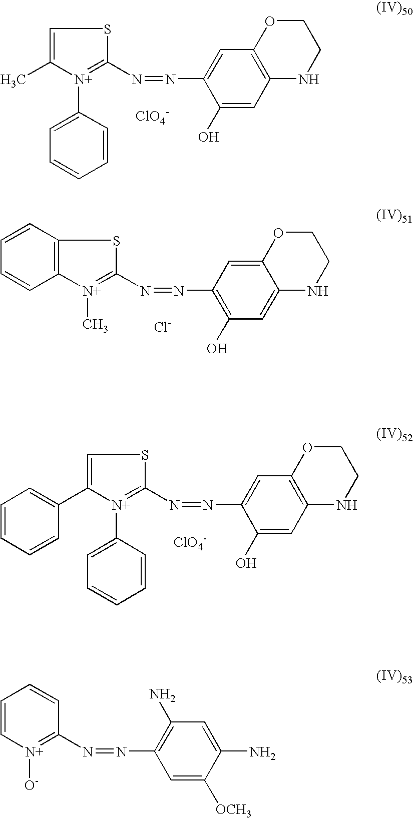

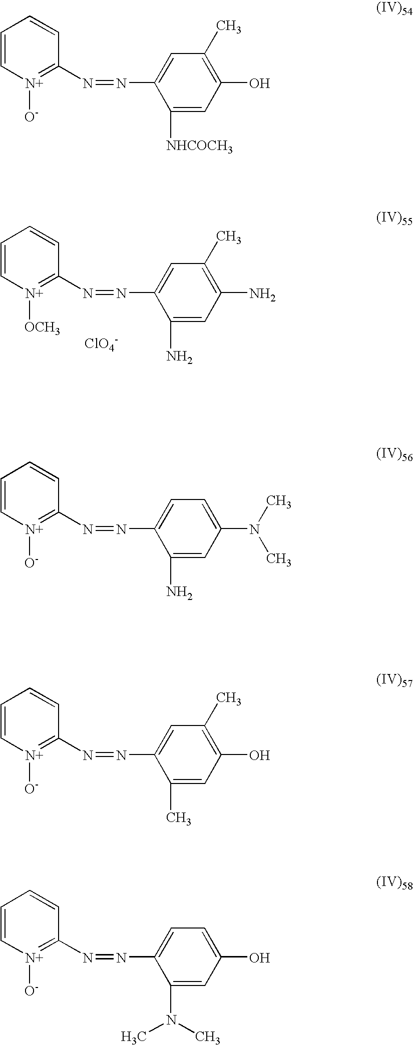

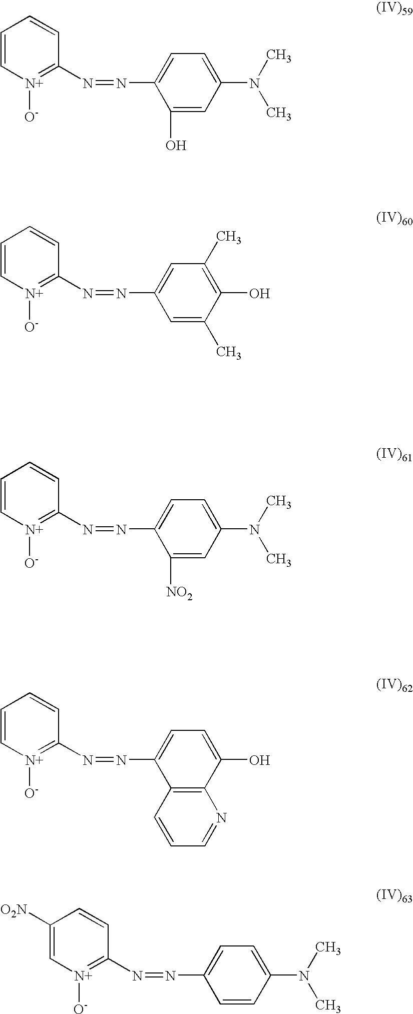

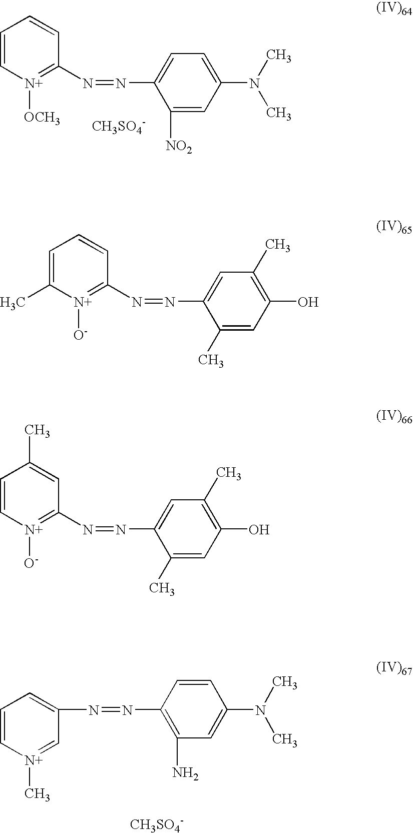

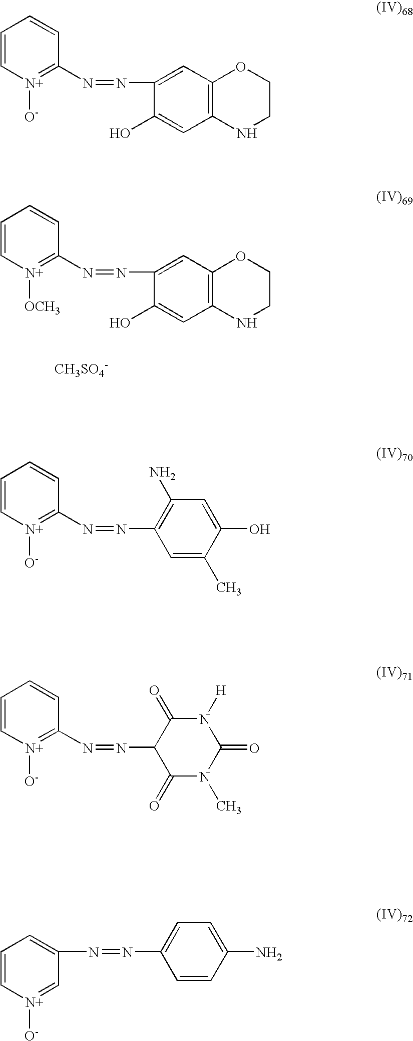

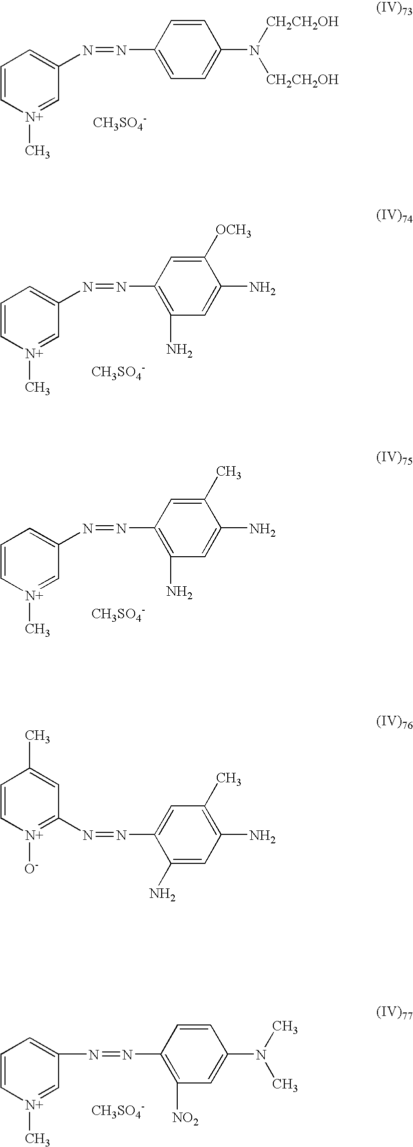

The cationic direct dye(s) used according to the invention preferably represent from 0.001 to 10% by weight approximately of the total weight of the dyeing composition and still more preferably from 0.005 to 5% by weight approximately of this weight.

(ii) The nonionic surfactant which can be used according to the present invention is chosen from the group consisting of:
(ii)₁—alkyl polyglucosides;
(ii)₂—sugar or alkyl sugar fatty acid esters;
(ii)₃—fatty sugar amides;
(ii)₄—mixtures thereof.

The nonionic surfactants of the alkyl glucoside type (ii), which are used in the present invention are well-known products per se which may be more particularly represented by the following general formula (V): 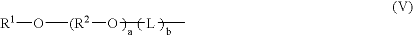
in which,

R¹ denotes a linear or branched alkyl and/or alkenyl radical comprising from about 8 to 24 carbon atoms, an alkylphenyl radical whose linear or branched alkyl group comprises from about 8 to 24 carbon atoms, R² denotes an alkylene radical comprising from 2 to 4 carbon atoms, L denotes a reduced sugar comprising from 5 to 6 carbon atoms, a denotes a value ranging from 0 to 10, and b denotes a value ranging from 1 to 15.

Preferred alkyl polyglucosides according to the present invention are compounds of formula (V) in which R¹ denotes more particularly a linear or branched alkyl and/or alkenyl radical comprising from 9 to 14 carbon atoms, a denotes a value ranging from 0 to 3 and still more particularly equal to zero, L denotes glucose, fructose or galactose. The degree of polymerization (S) of the saccharide, i.e. the value of b in formula (V), may range from 1 to 15. According to the invention, reduced sugars containing 80%, or more, of sugars whose degree of polymerization (S) takes a value ranging from 1 to 4 are preferred.

Compounds of formula (V) are in particular represented by the products sold by the company HENKEL under the name APG, such as the-products APG 300, APG 350, APG 500, APG 550, APG 625, APG base 10-12; the products sold by the company SEPPIC under the names TRITON CG 110 (or ORAMIX CG 110) and TRITON CG 312 (or ORAMIX NS 10); those sold by the company B.A.S.F. under the name LUTENSOL GD 70; those sold by the company HENKEL under the names PLANTAREN 1200, PLANTAREN 1300, PLANTAREN 2000 and PLANTACARE 2000, PLANTACARE 818, PLANTACARE 1200.

The nonionic surfactants of the sugar or alkyl sugar fatty acid ester type (ii)₂ used in the present invention are sugar or alkyl sugar C₄-C₂₂ fatty acid esters among which there may be mentioned in particular:

(ii₂) (a) (C₁-C₄)alkyl glucoside esters such as:

methyl glucoside monostearate, such as the product sold under the name GRILLOCOSE IS by the company GRILLOWERKE;

methyl glucoside sesquistearate, such as the product sold under the name GLUCATE SS by the company AMERCHOL;

6-ethylglucoside decanoate, such as the product sold under the name BIOSURF 10 by the company NOVO;

the mixture of mono- and dicocoate (82/7) of 6-ethylglucoside, such as the product sold under the name BIOSURF COCO by the company NOVO;

the mixture of mono- and dilaurate (84/8) of 6-ethylglucoside, such as the product sold under the name BIOSURF 12 by the company NOVO;

the butyl glucoside C₁₂-C₁₈ fatty acid monoesters such as butyl glucoside monococoate, such as the product sold under the names REWOPOL V3101 or REWOSAN V3101 and polyoxyethylenated butyl glucoside monococoate with 3 moles of ethylene oxide, such as the product sold under the name REWOPOL V3122 by the company REWO.

(ii)₂(b) glucose esters such as:

6-O-hexadecanoyl-α-D-glucose, 6-O-octanoyl-D-glucose, 6-O-oleyl-D-glucose, 6-O-linoleyl-D-glucose, which are known compounds which may be prepared, for example, from the corresponding acid chloride and D-glucose, according to the method described by E.REINEFELD et al., “Die Stärke” No. 6—pages 181-189,—1968.

(ii)₂(C) sucrose monoesters such as:

sucrose monolaurate, such as the product sold under the name GRILLOTEN LES 65, and

sucrose monococoate sold under the name GRILLOTEN LES 65K sold by the company GRILLO-WERKE.

The nonionic surfactants of the fatty sugar amide type (ii)₃ used in the present invention are compounds comprising at least one amide function and including at least one sugar or sugar derivative portion and at least one fatty chain; such compounds may, for example, result from the action of a fatty acid or a fatty acid derivative on the amine function of an amino sugar, or from the action of a fatty amine on a sugar comprising a carboxylic acid function (free or in lactone form) or carboxylic acid-derived function or alternatively a carbonyl function, and optionally in the presence of suitable co-reagents.

The fatty sugar amides (ii)₃ used are preferably chosen from:

(ii)₃(a) N-substituted aldonamides, and

(ii)₃(b) polyhydroxylated fatty acid amides or mixtures thereof.

(ii)₃(a) The N-substituted aldonamides which can be used according to the invention may be chosen from those described in patent application EP-A-550,106 whose content is an integral part of the present description. Among these, there may be mentioned:

N-substituted lactobionamides, N-substituted maltobionamides, N-substituted cellobionamides, N-substituted mellibionamides and N-substituted gentiobionamides such as:

(i) N-alkyllactobionamides, N-alkylmaltobionamides, N-alkylcellobionamides, N-alkylmellibionamides or N-alkylgentiobionamides which are mono- or disubstituted with a saturated or unsaturated, linear or branched, aliphatic hydrocarbon group which may contain heteroatoms preferably having up to 36 carbon atoms, more preferably up to 24 carbon atoms and still more particularly from 8 to 18 (for example methyl, ethyl, amyl, hexyl, heptyl, nonyl, decyl, undecyl, dodecyl, tridecyl, tetradecyl, pentadecyl, hexadecyl, heptadecyl, octadecyl; allyl, undecenyl, oleyl, linoleyl, propenyl, heptenyl), with an aromatic hydrocarbon group (for example benzyl, aniline, substituted benzyl, phenylethyl, phenoxyethyl, vinylbenzyl) or cycloaliphatic groups (for example cyclopentyl, cyclohexyl);

(ii) N-lactobionylamino acid esters where the amino acid may denote in particular: alanine, valine, glycine, lysine, leucine, arginine, aspartic acid, glutamic acid, threonine, serine, cysteine, histidine, tyrosine, methionine or which may be chosen, for example, from β-alanine, sarcosine, gamma-aminobutyric acid, ornithine, citrulline or their equivalents; the said N-lactobionylamino acid esters being monosubstituted with a group of formula: 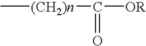
where R is an aliphatic hydrocarbon group which may contain up to 36 carbon atoms and n is an integer greater than 1, as well as the corresponding N-maltobionylamino acid esters, the N-mellibionylamino acid esters, the N-cellobionylamino acid esters and the N-gentiobionylamino acid esters;

(iii) N-(alkyloxy)alkyllactobionamides which are mono- or disubstituted with a group —(CH₂)ₙOR′ where R′ is an aliphatic, aromatic or cycloaliphatic hydrocarbon group as defined in paragraph (i);

(iv) N-(polyalkyloxy)alkyllactobionamides,

N-(polyalkyloxy)alkylmaltobionamides,

N-(polyalkyloxy)alkylcellobionamides,

N-(polyalkyloxy)alkylmellibionamides or

N-(polyalkyloxy)alkylgentiobionamides which are mono- or disubstituted with a group —R₁—(OR₁)ₙR₁R₂ where R₁ is an alkylene group such as ethylene, propylene or mixtures thereof, n is an integer greater than 1, R₂ is a lactobionamide, maltobionamide, cellobionamide, mellibionamide or gentiobionamide group.

(ii)₃(b) The polyhydroxylated fatty amides in accordance with the present invention are preferably chosen from those described in patent EP-B-550,656 whose content is an integral part of the description and corresponding to the following formula (VI): 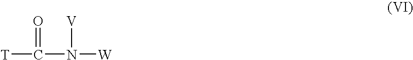
in which,

T denotes a C₅-C₃₁ hydrocarbon group, preferably a C₇-C₁₅ linear alkyl or alkenyl chain of mixtures thereof;

V denotes hydrogen, a C₁-C₄ hydrocarbon radical, 2-hydroxyethyl, 2-hydroxypropyl or mixtures thereof, preferably a C₁-C₄ alkyl such as methyl, ethyl, propyl, isopropyl, N-butyl and more particularly methyl;

W denotes a polyhydroxy hydrocarbon-containing group having a linear hydrocarbon chain with at least 3 hydroxyl groups directly attached to the chain, or an alkoxylated derivative of the said group (preferably ethoxylated or propoxylated).

W is preferably a reducing sugar derivative obtained by reductive amination reaction and more preferably a glycityl group. Glucose, maltose, lactose, galactose, mannose and xylose may be mentioned among the reducing sugars.

Still more preferably, W is chosen from the groups of the following formulae:
—(CH₂)—(CHOH)ₙ—CH₂OH;
—CH—(CH₂OH)—(CHOH)ₙ₋₁—CH₂OH;

—CH₂—(CHOH)₂(CHOR′) (CHOH)—CH₂OH in which n is an integer ranging from 3 to 5, R′ is hydrogen, a cyclic or aliphatic monosaccharide or one of its alkoxylated derivatives,

and among which a glycityl group in which n is equal to 4, and in particular the group —CH₂—(CHOH)₄—CH₂OH are again preferred.

The group T-CON═ may be for example cocamide, stearamide, oleamide, lauramide, myristiramide, capricamide, palmitamide, tallow amide.

According to the present invention, the use of the alkyl polyglucosides (ii)₁ as nonionic surfactants is more particularly preferred.

The nonionic surfactant(s) (ii) used according to the invention preferably represent from 0.05 to 30% by weight approximately of the total weight of the dyeing composition and still more preferably from 0.1 to 15% by weight approximately of this weight.

The appropriate dyeing medium (or carrier) generally consists of water or of a mixture of water and of at least one organic solvent for solubilizing the compounds which would not be sufficiently soluble in water. As organic solvent, there may be mentioned for example the C₁-C₄ lower alkanols such as ethanol and isopropanol, the aromatic alcohols such as benzyl alcohol as well as similar products and mixtures thereof.

in which W is a propylene residue which is optionally substituted with a hydroxyl group or a C₁-C₆ alkyl radical; R³, R⁴, R⁵ and R⁶, which are identical or different, represent a hydrogen atom, a C₁-C₆ alkyl radical or a C₁-C₆ hydroxyalkyl radical.

The dyeing composition in accordance with the invention may, in addition to the cationic direct dye(s) (i) defined above, contain one or more additional direct dyes which may for example be chosen from the nitrobenzene dyes, the anthraquinone dyes, the naphthoquinone dyes, the triarylmethane dyes, the xanthene dyes, the noncationic azo dyes.

When it is intended for oxidation dyeing, the dyeing composition in accordance with the invention contains, in addition to the cationic direct dye(s) (i), one or more oxidation bases chosen from the oxidation bases conventionally used for oxidation dyeing and among which there may be mentioned in particular the para-phenylenediamines, the bis-phenylalkylenediamines, the para-aminophenols, the ortho-aminophenols and the heterocyclic bases. When they are used, the oxidation base(s) preferably represent from 0.0005 to 12% by weight approximately of the total weight of the dyeing composition, and still more preferably from 0.005 to 6% by weight approximately of this weight.

When it is intended for oxidation dyeing, the dyeing composition in accordance with the invention may also contain, in addition to the cationic direct dye (i) and the nonionic surfactant (ii) as well as oxidation bases, one or more couplers so as to modify or increase the shimmer of the shades obtained using the cationic direct dye(s) (i) and the oxidation base(s).

The couplers which can be used in the dyeing composition in accordance with the invention may be chosen from the couplers conventionally used in oxidation dyeing and among which there may be mentioned in particular the meta-phenylenediamines, the meta-aminophenols, the meta-diphenols and the heterocyclic couplers.

When they are present, the coupler(s) preferably represent from 0.0001 to 10% by weight approximately of the total weight of the dyeing composition and still more preferably from 0.005 to 5% by weight approximately of this weight.

The dyeing composition in accordance with the invention may also contain various adjuvants which are conventionally used in hair-dyeing compositions, such as antioxidants, penetrating agents, sequestrants, perfumes, buffers, dispersing agents, film-forming agents, ceramides, preservatives, screening agents and opacifying agents.

Of course, persons skilled in the art will be careful to choose this or these optional additional compounds such that the advantageous properties intrinsically attached to the dyeing composition in accordance with the invention are not, or not substantially, altered by the addition(s) envisaged.

The dyeing composition according to the invention may be provided in various forms, such as in the form of liquids, shampoos, creams, gels, or in any other form appropriate for dyeing keratinous fibres, and in particular human hair. It may be obtained by freshly mixing a composition, which is optionally pulverulent, containing the cationic direct dye(s) with a composition containing the nonionic surfactant.

When the combination of the cationic direct dye (i) and of the nonionic surfactant (ii) according to the invention is used in a composition intended for oxidation dyeing (one or more oxidation bases are then used, optionally in the presence of one or more couplers) or when it is used in a composition intended for direct lightening dyeing, then the dyeing composition in accordance,-with the invention contains, in addition, at least one oxidizing agent chosen for example from hydrogen peroxide, urea peroxide, alkali metal bromates, persalts such the perborates and persulphates, and enzymes such as peroxidases, laccases and oxidoreductases containing two electrons. The use of hydrogen peroxide or of enzymes is particularly preferred.

Another subject of the invention is a method of dyeing keratinous fibres and in particular human keratinous fibres such as hair using the dyeing composition as defined above.

According to a first variant of this dyeing method in accordance with the invention, at least one dyeing composition as defined above is applied to the fibres for a sufficient time to develop the desired colour, after which they are rinsed, optionally washed with shampoo, rinsed again and dried.

The time necessary for the development of the colour on the keratinous fibres is generally between 3 and 60 minutes and still more preferably 5 and 40 minutes.

According to a second variant of this dyeing method in accordance with the invention, at least one dyeing composition as defined above is applied to the fibres for a sufficient time to develop the desired colour, with no final rinsing.

According to a particular embodiment of this dyeing method, and when the dyeing composition in accordance with the invention contains at least one oxidation base and at least one oxidizing agent, the dyeing method comprises a preliminary stage consisting of storing in a separate form, on the one hand, a composition (A1) comprising, in an appropriate dyeing medium, at least one cationic direct dye (i) as defined above and at least one oxidation base and, on the other hand, a composition (B1) containing, in an appropriate dyeing medium, at least one oxidizing agent, and then mixing them at the time of use before applying this mixture to the keratinous fibres, the composition (A1) or the composition (B1) containing the nonionic surfactant (ii) as defined above.

According to another particular embodiment of this dyeing method, and when the dyeing composition in accordance with the invention contains at least one oxidizing agent, the dyeing method comprises a preliminary stage consisting of storing in a separate form, on the one hand, a composition (A2) comprising, in an appropriate dyeing medium, at least one cationic direct dye (i) as defined above and, on the other hand, a composition (B2) containing, in an appropriate dyeing medium, at least one oxidizing agent, and then mixing them at the time of use before applying this mixture to the keratinous fibres, the-composition (A2) or the composition (B2) containing the nonionic surfactant as defined above.

Another subject of the invention is a multicompartment device or dyeing “kit” or any other multicompartment packaging system in which a first compartment contains composition (A1) or (A2) as defined above and a second compartment contains composition (B1) or (B2) as defined above. These devices may be equipped with a means allowing the desired mixture to be delivered to the hair, such as the devices described in patent FR-2,586,913 in the applicant's name.

The following examples are intended to illustrate the invention without, however, limiting the scope thereof.

### EXAMPLES

### Examples 1 to 9

The nine direct dyeing compositions which are assembled in the following table were prepared:

(all contents expressed in grams)

| | EXAMPLES NO. | | | | | | | | |
|---|---|---|---|---|---|---|---|---|---|
| | 1 | 2 | 3 | 4 | 5 | 6 | 7 | 8 | 9 |
| Cationic direct dye of formula (IV)₁₀ | 0.12 | | | | | | | | |
| Cationic direct dye of formula (IV)₂₇ | | 0.10 | | | | | | | |
| Cationic direct dye of formula (IV)₁₇ | | | 0.10 | | | | | | |
| Cationic direct dye of formula (IV)₄₁ | | | | 0.11 | | | | | |
| Cationic direct dye of formula (IV)₂₅ | | | | | 0.12 | | | | |
| Cationic direct dye of formula (I 1) | | | | | | 0.10 | | | 0.10 |
| Cationic direct dye of formula (I 2) | | | | | | 0.10 | | | |
| Cationic direct dye of formula (I 14) | | | | | | | 0.20 | | |
| Cationic direct dye of formula (I 31) | | | | | | | | 0.15 | |
| Alkyl polyglucoside sold under the name | | 8.0 | 8.0 | | | | | | 8.0 |
| ORAMIX CG110 by the company SEPPIC | | | | | | | | | |
| N-decanoyl-N-methylglucamine** | 8.0 | | | | | | 8.0 | | |
| 6-O-hexadecanoyl-α-D-glucose | | | | 8.0 | | 8.0 | | | |
| N-cocolactobionamide | | | | | 8.0 | | | 8.0 | |
| Ethanol | 10 | 10 | 10 | 10 | 10 | 10 | 10 | 10 | 10 |
| 2-amino-2-methyl-1-propanol qs | pH 9 | pH 9 | pH 9 | pH 9 | pH 9 | pH 9 | pH 9 | pH 9 | pH 9 |
| Demineralized water qs | 100 | 100 | 100 | 100 | 100 | 100 | 100 | 100 | 100 |

**N-decanoyl-N-methylglucamine [polyhydroxylated fatty acid amide of formula: C₉H₁₉—CO—N(CH₃)—CH₂—(CHOH)₄—CH₂OH]

The above compositions were each applied for 30 minutes to locks of natural grey hair which is 90% white. The hair locks were then rinsed, washed with a standard shampoo and then dried.

The locks were dyed in the following shades:

| Examples | Shades obtained |
|---|---|
| 1 | dark red |
| 2 | dark purple |
| 3 | dark orange |
| 4 | dark red |
| 5 | dark orange |
| 6 | dark red |
| 7 | dark orange |
| 8 | dark violet |
| 9 | dark red |

1-39. (canceled)

40. A composition for dyeing keratinous fibers comprising
(i) at least one cationic direct dye chosen from compounds having the following formulae (I), (II), (III), (III′), or (IV):
a) compounds of following formula (I): 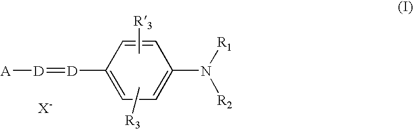
in which:
D is a nitrogen atom or a —CH group,
R₁ and R₂, which are identical or different, are chosen from a hydrogen atom; a (C₁-C₄) alkyl radical which may be unsubstituted or substituted with a BCN radical, an —OH radical or an —NH₂ radical or form with each other or with a carbon atom of the benzene ring an optionally oxygen-containing or nitrogen-containing heterocycle which may be unsubstituted or substituted with at least one radical chosen from (C₁-C₄) alkyl radicals; and a 4=-aminophenyl radical,
R₃ and R=₃, which are identical or different, are chosen from a hydrogen atom; a halogen atom chosen from chlorine, bromine, iodine, and fluorine; a cyano group; a (C₁-C₄) alkyl radical; a (C₁-C₄) alkoxy radical; and a (C₁-C₄) acetyloxy radical,
X⁻ is an anion,
A is chosen from the following structures A₁ to A₁₉: 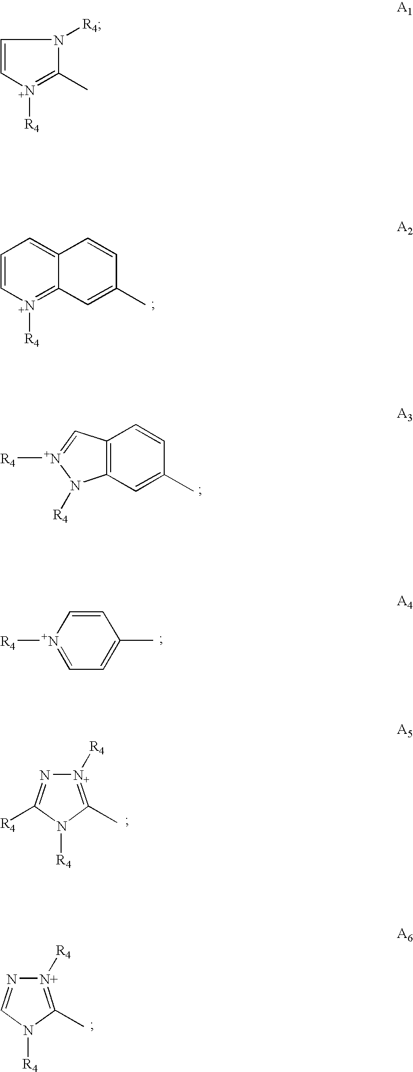 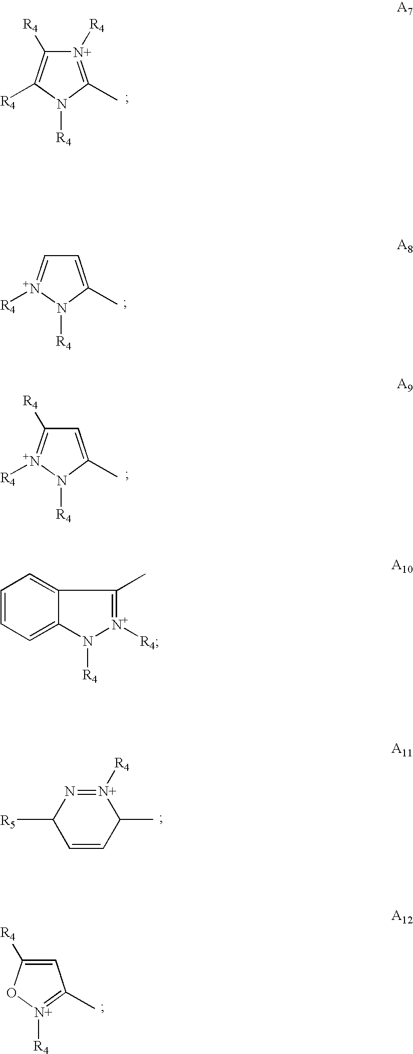 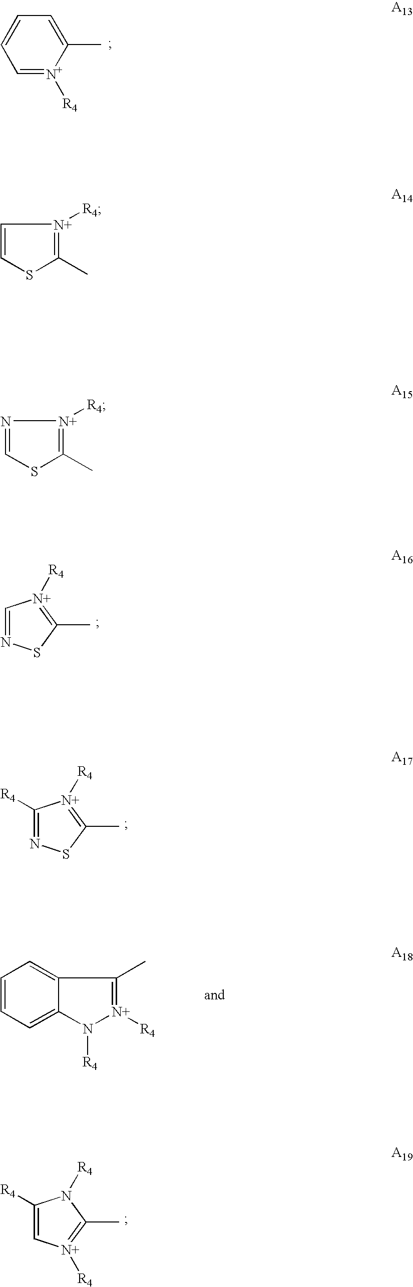
in which:
R₄ is a (C₁-C₄) alkyl radical which may be unsubstituted or substituted with a hydroxyl radical, and
R₅ is a (C₁-C₄) alkoxy radical,
with the proviso that when D is a BCH group, A is A₄ or A₁₃ and R₃ is different from a (C₁-C₄) alkoxy radical, then R₁ and R₂ are not simultaneously a hydrogen atom;
b) compounds of following formula (II): 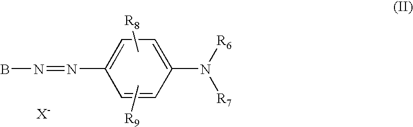
in which:
R₆ is a hydrogen atom or a (C₁-C₄) alkyl radical,
R₇ is chosen from a hydrogen atom, an alkyl radical which may be unsubstituted or substituted with a —CN radical or with an amino group, and a 4=-aminophenyl radical, or forms, with R₆, a heterocycle which optionally contains at least one heteroatom chosen from oxygen and nitrogen and wherein said heterocycle may be unsubstituted or substituted with at least one radical chosen from (C₁-C₄) alkyl radicals,
R₈ and R₉, which are identical or different, are chosen from a hydrogen atom, a halogen atom chosen from bromine, chlorine, iodine or fluorine, a (C₁-C₄) alkyl radical, a (C₁-C₄) alkoxy radical, and a —CN radical,
X⁻ is an anion,
B is a group chosen from the following structures B1 to B6: 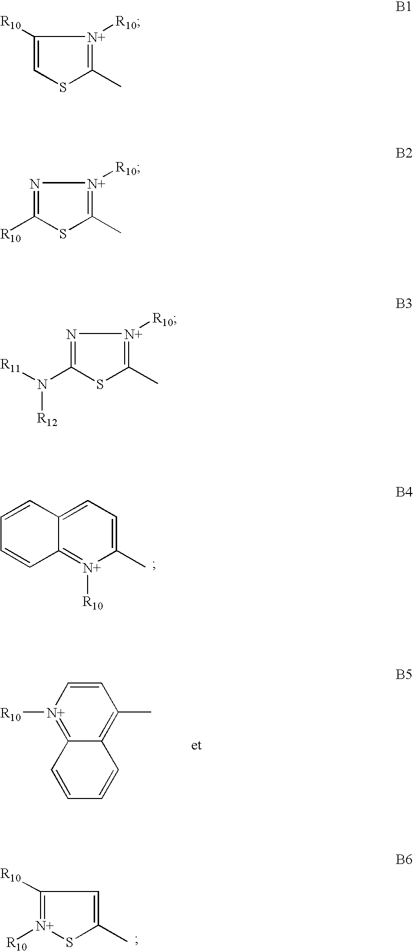
in which:
R₁₀ is a (C₁-C₄) alkyl radical,
R₁₁ and R₁₂, which are identical or different, are a hydrogen atom or a (C₁-C₄) alkyl radical;
c) compounds of following formulae (III) and (III′): 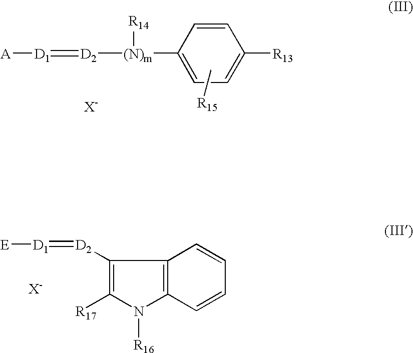
in which:
R₁₃ is chosen from a hydrogen atom, a (C₁-C₄) alkoxy radical, a halogen atom chosen from bromine, chlorine, iodine and fluorine atoms, and an amino radical,
R₁₄ is chosen from a hydrogen atom, and a (C₁-C₄) alkyl radical, or forms, with a carbon atom of the benzene ring, a heterocycle which optionally contains an oxygen heteroatom and which is unsubstituted or substituted with at least one radical chosen from (C₁-C₄) alkyl radicals,
R₁₅ is a hydrogen atom or a halogen atom chosen from bromine, chlorine, iodine and fluorine,
R₁₆ and R₁₇, which are identical or different, are a hydrogen atom or a (C₁-C₄) alkyl radical,
D₁ and D₂, which are identical or different, are a nitrogen atom or a —CH group,
m is 0 or 1,
with the proviso that when R₁₃ is an unsubstituted amino group, then D₁ and D₂ simultaneously are a —CH group and m=0,
X⁻ is an anion,
E is a group chosen from the following structures E1 to E8: 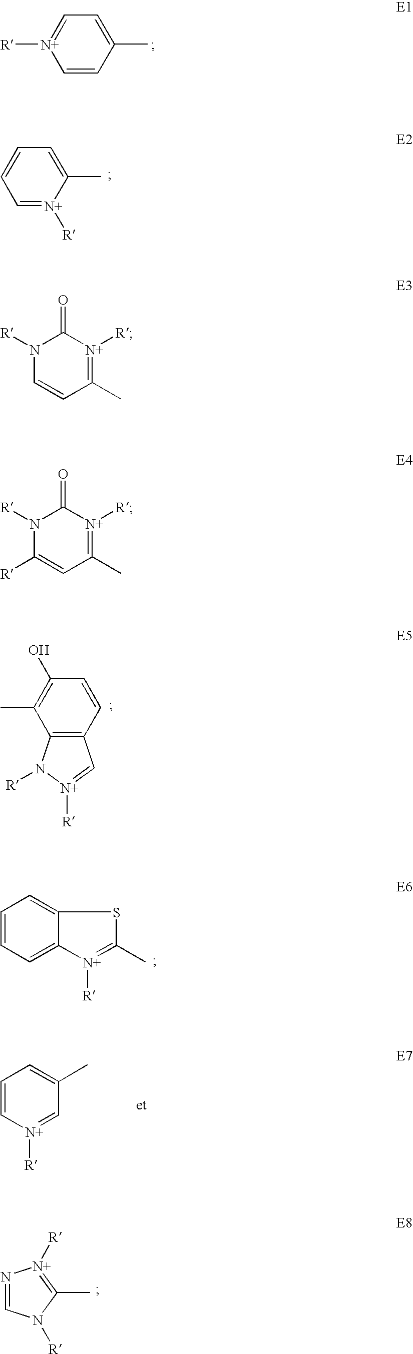
in which R═ is a (C₁-C₄) alkyl radical;
when m is 0 and D₁ is a nitrogen atom, then E may also be a group having the following structure E9: 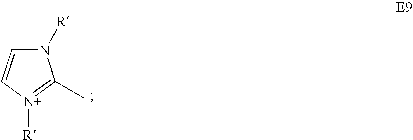
in which R═ is a (C₁-C₄) alkyl radical,
d) compounds of following formula (IV): 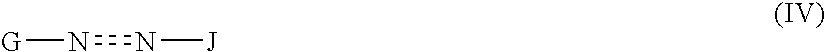
in which:
G is a group chosen from the following structures G₁ to G₃: 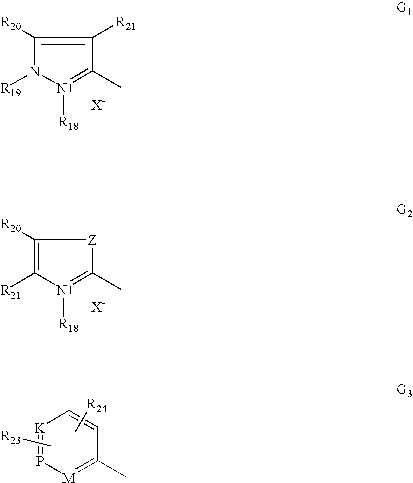
in which,
R₁₈ is chosen from a (C₁-C₄) alkyl radical, a phenyl radical which may be unsubstituted or substituted with at least one radical chosen from (C₁-C₄) alkyl radicals, and a halogen atom chosen from chlorine, bromine, iodine and fluorine;
R₁₉ is a (C₁-C₄) alkyl radical or a phenyl radical;
R₂₀ and R₂₁, which are identical or different, are chosen from a (C₁-C₄) alkyl radical and a phenyl radical; or form together in G₁ a benzene ring which is substituted with at least one radical chosen from (C₁-C₄) alkyl radicals, (C₁-C₄) alkoxy radicals, and NO₂ radicals; or form together in G₂ a benzene ring which is unsubstituted or substituted with at least one radical chosen from (C₁-C₄) alkyl radicals, (C₁-C₄) alkoxy radicals, and NO₂ radicals;
R₂₀ may also be a hydrogen atom;
Z is chosen from an oxygen atom, a sulphur atom, and an —NR₁₉ group;
M, K, and P, which are identical or different, are chosen from a
BCH group, a BC(C₁-C₄ alkyl) group, and a —NR₂₂(X⁻)ᵣ group;
r is zero or 1;
R₂₂ is chosen from an O⁻ atom, a (C₁-C₄) alkoxy radical, and a (C₁-C₄) alkyl radical;
R₂₃ and R₂₄, which are identical or different, are chosen from a hydrogen atom, a halogen atom chosen from chlorine, bromine, iodine and fluorine, a (C₁-C₄) alkyl radical, a (C₁-C₄) alkoxy radical, and an —NO₂ radical;
X⁻ is an anion;
the symbol J is chosen from:
(a) a group having the following structure J₁: 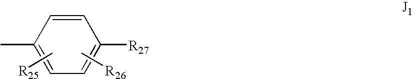
in which,
R₂₅ is a hydrogen atom; a halogen atom chosen from chlorine, bromine, iodine, and fluorine; a (C₁-C₄) alkyl radical; a (C₁-C₄) alkoxy radical; an BOH radical, an —NO₂ radical, a —NHR₂₈ radical, an —NR₂₉R₃₀ radical, and a —NHCO(C₁-C₄alkyl) radical, or forms, with R₂₆, a 5- or 6-membered ring optionally containing at least one heteroatom chosen from nitrogen, oxygen, and sulphur;
R₂₆ is chosen from a hydrogen atom; a halogen atom chosen from chlorine, bromine, iodine, and fluorine; a (C₁-C₄) alkyl radical; and a (C₁-C₄ ) alkoxy radical, or forms with R₂₇ or R₂₈ a 5- or 6-membered ring optionally containing at least one heteroatom chosen from nitrogen, oxygen, and sulphur;
R₂₇ is chosen from a hydrogen atom, an —OH radical, an —NHR₂₈ radical, and an —NR₂₉R₃₀ radical;
R₂₈ is chosen from a hydrogen atom, a (C₁-C₄) alkyl radical, a (C₁-C₄) monohydroxyalkyl radical, a (C₂-C₄) polyhydroxyalkyl radical, and a phenyl radical;
R₂₉ and R₃₀, which are identical or different, are chosen from a (C₁-C₄) alkyl radical, a (C₁-C₄) monohydroxyalkyl radical, and a (C₂-C₄) polyhydroxyalkyl radical; and
(b) wherein J is further chosen from a 5- or 6-membered nitrogen-containing heterocycle group which optionally contains at least one heteroatom and/or at least one carbonyl-containing group and wherein said heterocycle may be unsubstituted or substituted with at least one radical chosen from (C₁-C₄) alkyl radicals, an amino radical and a phenyl radical; and
wherein said composition further comprises
(ii) at least one nonionic surfactant chosen from:
(ii)₁—alkyl polyglucosides;
(ii)₂—sugar or alkyl sugar fatty acid esters; and
(ii)₃—fatty sugar amides.
